(19) United States
(12) Patent Application Publication
(10) Pub. No.: US 2022/0296749 A1
(43) Pub. Date: Sep. 22, 2022

(54) SCISSORS HOLDER HAVING STERILIZATION FUNCTION

(71) Applicant: Dong Ho JUN, Seoul (KR)

(72) Inventor: Dong Ho JUN, Seoul (KR)

(21) Appl. No.: 17/684,521

(22) Filed: Mar. 2, 2022

(30) Foreign Application Priority Data
Mar. 19, 2021 (KR) 10-2021-0036275

(51) Int. Cl. A61L 2/10; A61L 2/26

(52) U.S. Cl. CPC A61L 2/10; A61L 2/26; A61L 2202/11

(57) ABSTRACT

Disclosed herein is a scissors holder having a sterilization function. The scissors holder includes a loading part including a plurality of holding holes having a width larger than that of the blades of scissors and smaller than that of the handles of the scissors so that the scissors can be held therein in such a manner that the blades of the scissors enter thereinto and the handles of the scissors are supported thereon, a support part disposed on both lateral sides of the loading part so that the loading part can be disposed to be spaced apart from the ground or a surface of a piece of furniture by a predetermined distance, an internal space defined by the bottom of the loading part and the support part, and a sterilization part configured to sterilize the blades of scissors held in the internal space.

### CROSS-REFERENCE TO RELATED APPLICATION

This application claims the benefit of Korean Patent Application No. 10-2021-0036275 filed on Mar. 19, 2021, which is hereby incorporated by reference herein in its entirety.

### BACKGROUND

### 1. Technical Field

The present invention relates generally to a scissors holder having a sterilization function, and more particularly to a scissors holder having a sterilization function, which includes a loading part including a plurality of holding holes having a width larger than that of the blades of scissors and smaller than that of the handles of the scissors so that the scissors can be held therein in such a manner that the blades of the scissors enter thereinto and the handles of the scissors are supported thereon, a support part disposed on both lateral sides of the loading part so that the loading part can be disposed to be spaced apart from the ground or a surface of a piece of furniture by a predetermined distance, and a sterilization part including an internal space defined by the bottom of the loading part and the support part and configured to sterilize the blades of scissors held in the internal space.

### 2. Description of the Related Art

The description given in this section merely provides background information about the present invention, and does not constitute the prior art.

Paragraph 1 of Article 4 of the Korean Public Health Control Act stipulates that beauty instruments such as hairdressing scissors, hair brushes, and electric clippers used in beauty salons should be used only for one customer after sanitary sterilization.

However, as regards the current states of actual beauty salons, a fixed UV sterilizer is provided in a designated place to obtain permission to open a beauty salon regardless of the size of the salon and the number of professional hairdressers, and there are considerably few cases where sanitary sterilization is performed in practice.

According to an investigation made by a media outlet, 50 to 850,000 CFU (a colony forming unit)/10 cm² and 75 to 1,200,000 CFU/10 cm² of mold, bacteria, or bacteria such as Demodex and Staphylococcus aureus were detected in hairdressing scissors and hairbrushes, respectively, that were not sterilized at beauty salons.

These are significantly high detection values compared to the normal bacterial detection value of 10 to 3,300 CFU/10 cm² detected in the handles of toilets. Accordingly, when a beauty procedure is performed using a beauty instrument that is not appropriately sterilized, there is a risk of infection with various types of dermatitis.

In particular, as the interest of people today in their appearances increases, the number of visits to beauty salons increases. Further, since beauty instruments come into direct contact with the human bodies during beauty work and beauty instruments are used for an unspecified number of people, there is a risk of bacterial infection. Accordingly, it is necessary to thoroughly prepare for the hygiene management of various types of beauty instruments.

In order to overcome these anxiety factors, according to a conventional UV charging sterilizer for hairdressing instruments (Korean Patent Application Publication No. 10-2010-0051598), an auxiliary material storage part configured to store perm paper, a hairbrush storage part and a hair clipper storage part disposed on both sides of the auxiliary material storage part and configured to accommodate hairbrushes and hair clippers, respectively, and a plurality of hairdressing scissors storage parts and razor storage parts disposed in front of the auxiliary material storage part and configured to accommodate a plurality of types of hairdressing scissors and razors, respectively, are provided on the top surface thereof, and a sterilizer housing made of transparent or opaque synthetic resin is also provided.

However, since there is a sufficient possibility that the beauty instruments placed in a holder in a sterilized state may be contaminated again during use, there is a lot of difficulty in allowing the beauty instruments stored in the holder to always be maintained in a sterilized state in conventional beauty salons.

In order to solve the problems of the conventional beauty instrument holders described above, some domestic and foreign related companies had conducted research on beauty instrument holders equipped with a sterilization function. However, these beauty instrument holders have lower market competitiveness than the conventional beauty instrument holders because the cost required to provide a structure having a sterilization function is excessively high for the purpose of actual commercialization or a sterilization effect is not sufficiently high compared to the fabrication cost of the apparatuses. As a result, there were not actually commercialized cases.

Therefore, there is a demand for the development of an apparatus capable of solving the problems of the conventional technology described above.

### SUMMARY

The present invention has been conceived to overcome the above-described problems, and objects of the present invention are as follows:

A first object of the present invention is to provide a scissors holder that includes an internal space capable of minimizing the entry of external materials defined by the bottom of a loading part and a support part and sterilizes beauty instruments via an ultraviolet C (UV-C) radiation unit disposed in the internal space and an UV-C radiation unit disposed above the loading part and attached to one side of the loading part, thereby maintaining hygiene and cleanliness.

A second object of the present invention is to provide a scissors holder that is provided with a reflector including a first panel, a second panel, and a third panel in order to prevent the light, radiated from a first radiation unit, from being radiated onto the outside.

The objects of the present invention are not limited to the objects described above, and other objects not described above will be clearly understood by those of ordinary skill in the art from the following description.

According to an aspect of the present invention, there is provided a scissors holder having a sterilization function, the scissors holder including: a loading part including a plurality of holding holes each having a width larger than that of the blades of scissors and smaller than that of the handles of the scissors so that the scissors can be held therein in such a manner that the blades of the scissors go into the holding holes and the handles of the scissors are supported on the loading part; a support part disposed on both lateral sides of the loading part so that the loading part can be disposed to be spaced apart from the ground or a surface of a piece of furniture by a predetermined distance; an internal space defined by the bottom of the loading part and the support part; and a sterilization part configured to sterilize the blades of scissors held in the internal space.

Each of the holding holes may include: a first holding hole having a width larger than that of the blades of entering scissors so that the blades of the entering scissors can enter thereinto; and a second holding hole disposed above the first holding hole and having a width larger than that of the first holding hole in order to guide the blades of the scissors through entry into the first holding hole.

The width of the second holding hole may be gradually increased from the bottom portion thereof toward the top surface thereof in order to guide the blades of the scissors in a closed state through entry into the first holding.

The sterilization part may include: one or more first radiation units disposed on the bottom surface of the loading part to sterilize the blades of scissors, and configured to radiate ultraviolet (UV) rays; and one or more reflectors disposed on the bottom surface of the loading part to prevent light, radiated from the first radiation units, from being radiated onto the outside, and having a desired angle toward the internal space.

Each of the reflectors may include: a first panel formed to extend from the bottom surface of the loading part; a second panel formed to extend from one side of the first panel in a curved shape; and a third panel formed to extend from the other side of the second panel in a straight line shape.

The reflector may be disposed on each of the front and rear sides of the bottom surface of the loading part, or may further be disposed on each of both lateral sides of the bottom surface of the loading part.

The top surfaces of the support part may be inclined to support the loading part at a predetermined inclined angle.

The scissors holder may further include a second radiation unit configured to radiate UV rays toward scissors supported and loaded on the loading part to sterilize the handles of the scissors and disposed above the loading part and attached to one side of the loading part.

The first radiation units and the second radiation unit may radiate ultraviolet C (UV-C) light in the wavelength range of 100 to 280 nm.

The scissors holder may further include a variable portion coupled to one side of the second radiation unit to adjust the position of the second radiation unit and configured to be selectively changed in the shape thereof.

The second radiation unit may include a reflector having a desired angle toward a light source disposed inside the second radiation unit in order to prevent the light, radiated from the second radiation unit, from being radiated onto the outside and also allow the light to be focused toward the handles of scissors.

The second radiation unit may be coupled to the loading part to be freely detachable therefrom.

### BRIEF DESCRIPTION OF THE DRAWINGS

The above and other objects, features, and advantages of the present invention will be more clearly understood from the following detailed description taken in conjunction with the accompanying drawings, in which:

FIG. 1 is a perspective view of a scissors holder according to an embodiment of the present invention;

FIG. 2 is a detailed view of a holding hole according to an embodiment of the present invention;

FIG. 3 is a detailed view of a support part according to an embodiment of the present invention;

FIG. 4 is a rear view of a loading part according to an embodiment of the present invention; and

FIG. 5A and FIG. 5B are, respectively, a detailed view of a reflector according to an embodiment of the present invention.

### DETAILED DESCRIPTION

Specific embodiments of the present invention will be described in detail below with reference to the accompanying drawings.

However, in the following description of the specific embodiments of the present invention, when it is determined that a detailed description of a related known function or configuration may unnecessarily obscure the gist of the present invention, the detailed description will be omitted.

The above-described objects, features and advantages of the present invention will become more apparent from the following detailed description taken in conjunction with the accompanying drawings. However, the present invention may be subject to various modifications, and may include various embodiments. The specific embodiments will be illustrated in the accompanying drawings and described in detail below.

Furthermore, the numerals used in the description of the present specification are merely identification symbols each adapted to distinguish one component from other components.

Furthermore, the suffix “part” for the components used in the following description is used merely to facilitate the preparation of the specification, and does not have a distinctive meaning or role by itself.

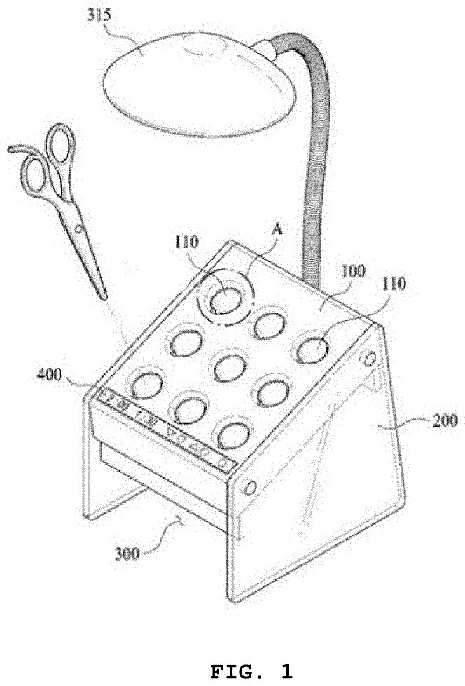

FIG. 1 is a perspective view of a scissors holder according to an embodiment of the present invention.

The scissors holder according to the present embodiment may include a loading part 100, a support part 200, a sterilization part 300, and a display part.

The loading part 100 may include a plurality of holding holes 110 having a width larger than the width of the blades of scissors and smaller than the width of the handles of the scissors so that the hairdressing scissors can be held therein in such a manner that the blades of the scissors go into the holding holes and the handles of the scissors are supported on the loading part 100 without going into the holding holes.

The support part 200 may be disposed on both lateral sides of the loading part 100 so that the loading part 100 can be disposed to be spaced apart from the ground or the surface of a piece of furniture by a predetermined distance.

An internal space is defined by the bottom of the loading part 100 and the support part 200, and the sterilization part 300 may sterilize the blades of scissors held in the internal space.

As a result, the internal space that may minimize the entry of external materials is formed by the bottom of the loading part 100 and the support part 200 and is fabricated as a minimum configuration for the sterilization of beauty instruments, so that a user's convenience of use may be improved.

The control module 400 may be disposed on one side of the loading part 100, and may control the power on/off, timer, and scheduled operation of the sterilization part 300, and may display buttons corresponding to functions to the user.

The power required for the sterilization part 300 and the control module 400 may be supplied by a power unit (not shown), and a known power supply means (e.g., a means for receiving power via a charging port in a wired manner, or a means for connecting an electric wire such as a USB cord, performing charging, and supporting wireless actual use) may be adopted as the power unit.

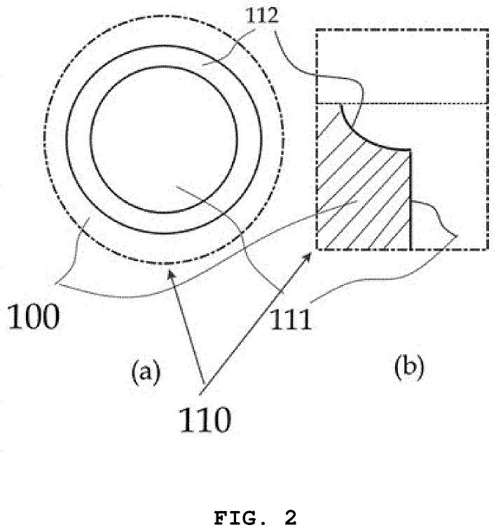

FIG. 2 is a detailed view of each of the holding holes 110 according to an embodiment of the present invention.

The holding hole 110 may include a first holding hole 111, and a second holding hole 112.

The first holding hole 111 may have a width larger than that of the blades of entering scissors so that the blades of the entering scissors can enter thereinto.

The second holding hole 112 may be disposed above the first holding hole 111 and have a width larger than that of the first holding hole 111 in order to guide the blades of scissors through entry into the first holding hole 111.

In addition, the width of the second holding hole 112 may be gradually increased from a bottom portion thereof, which is connected to the first holding hole 111, toward a top portion thereof in order to guide the closed blades of scissors through entry into the first holding hole 110.

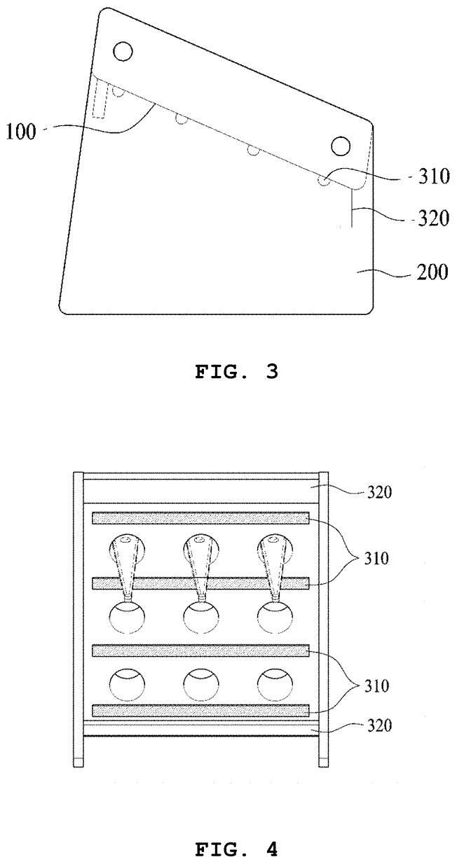

FIG. 3 is a detailed view of the support part 200 according to an embodiment of the present invention.

The top surfaces of the support part 200 may be inclined to support the loading part 100 at a predetermined inclined angle.

An ultraviolet (UV) blocking coating may be applied to the surfaces of the support part 200 in order to prevent the UV rays, radiated by first radiation units 310, from leaking to the outside.

The support part 200 may be composed of a rigid body that does not change in the shape thereof (for example, glass, metal such as iron, aluminum or copper, or synthetic resin such as polystyrene (PS), ABS, polyacetal (POM), polyethylene (PE), polyvinyl chloride (PVC), polycarbonate (PC), polycaprolactone (PCL) or polypropylene (PP) may be selected as the rigid body).

FIG. 4 is a rear view of the loading part 100 according to an embodiment of the present invention.

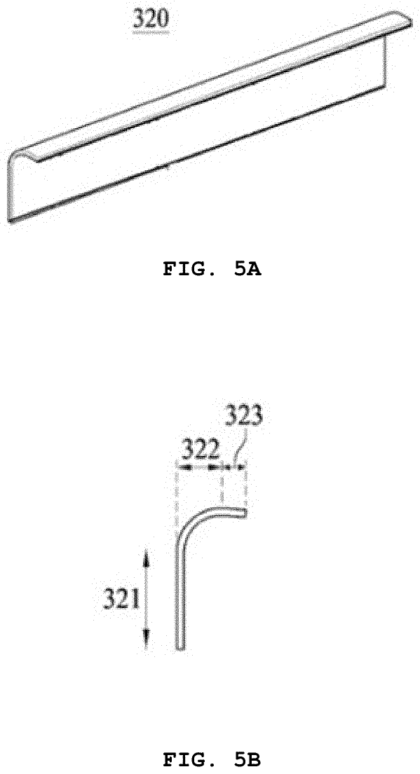

FIG. 5A and FIG. 5B are, respectively, a detailed view of a reflector 320 according to an embodiment of the present invention.

The sterilization part 300 according to an embodiment of the present invention may include the first radiation units 310, a second radiation unit 315, and reflectors 320.

The first radiation units 310 may be disposed on the bottom surface of the loading part 100 to sterilize the blades of scissors, and may radiate UV rays.

The first radiation units 310 and the second radiation unit 315 may radiate light in a wavelength range of 100 to 280 nm.

Although the first radiation units 310 may be formed on the rear surface of the loading part 100 on sides of horizontal rows in which the holding holes 110 are formed as shown in FIG. 4, the first radiation units 310 may be partially formed on sides of horizontal rows at the outermost ends, or may be formed on sides of vertical columns in which the holding holes 110 are formed.

The second radiation unit 315 may radiate ultraviolet rays toward the scissors loaded in the loading part 100 to sterilize the handles of the scissors, and may be disposed on one side of the loading part 100.

The second radiation unit 315 may include a variable portion.

The variable portion may be coupled to one side of the second radiation unit 315 to adjust the position of the second radiation unit 315, and may be selectively changed in the shape thereof.

The second radiation unit 315 may include a reflector having a desired angle toward a location below the second radiation unit 315 in order to prevent the light, radiated from the second radiation unit 315, from being radiated onto the outside other than the loading part and then being directly radiated onto a hairdresser and also allow the light to be focused toward the handles of scissors.

More specifically, the scissors holder according to an embodiment of the present invention may sterilize the blades of scissors disposed below the loading part 100 via the first radiation units 310, and may sterilize the handles of the scissors disposed above the loading part 100 via the second radiation unit 315. In other words, the overall surfaces of scissors are sterilized, so that the scissors holder according to the embodiment of the present invention may improve hygiene and cleanliness compared to the conventional scissors holders.

As shown in FIGS. 5A and 5B, reflector 320 may include a first panel 321, a second panel 322, and a third panel 323.

The reflector 320 may be disposed on the bottom surface of the loading part 100 to prevent the light, radiated from the first radiation units 310, from being radiated onto the outside, and may have a desired angle toward the internal space.

The first panel 321 may be formed to extend from the bottom surface of the loading part 100.

The second panel 322 may be formed to extend from one side of the first panel 321 in a curved shape.

The third panel 323 may be formed to extend from the other side of the second panel 322 in a straight line shape.

The reflector 320 is disposed on each of the front and rear sides of the bottom surface of the loading part 100, and may be further disposed on each of both the lateral sides of the bottom surface of the loading part 100.

More specifically, the reflectors 320 may be attached to the outer periphery of the bottom surface of the loading part 100 so that the light radiated from the first radiation units 310 can be focused toward the internal space without leaking to the sides or outside. As a fixing means for the reflectors 320, a known means, such as a method of applying an adhesive to a surface or a press fit method, may be adopted.

Although the reflector 320 including the first panel 321, the second panel 322, and the third panel 323 may be fabricated by being injected in an integrated form, it may be fabricated in a continuous form by combining and fixing some panels after injecting them in order to reduce fabrication difficulty, required fabrication time, and fabrication costs.

The effects of the present invention configured as described above will be described as follows:

A first effect of the present invention is to provide the internal space capable of minimizing the entry of external materials defined by the bottom of the loading part and the support part and sterilize beauty instruments via the UV-C radiation units disposed in the internal space and the UV-C radiation unit disposed above the loading part and attached to one side of the loading part, thereby maintaining hygiene and cleanliness.

A second effect of the present invention is to be fabricated as a minimum configuration for the sterilization of beauty instruments and align all the loading directions of beauty instruments that are held in the holding holes, thereby improving a user's convenience of use.

A third effect of the present invention is to prevent the light, radiated from the first radiation units, from being radiated onto the outside.

The effects of the present invention are not limited to the objects described above, and other effects not described above will be clearly understood by those of ordinary skill in the art from the foregoing description.

The above embodiments are merely illustrative of the technical spirit of the present invention, and those of ordinary skill in the art to which the present invention pertains may make various modifications and variations of these embodiments without departing from the essential features of the present invention.

The embodiments are not intended to limit the technical spirit of the present invention but are intended to describe the technical spirit of the present invention, and therefore, the scope of the present invention is not limited by these embodiments.

The scope of the present invention should be defined based on the claims, and all technical spirits that are equal or equivalent to the claims should be construed as being included in the scope of the present invention.

What is claimed is:

1. A scissors holder having a sterilization function, the scissors holder comprising:
a loading part configured to hold scissors, the loading part including holding holes each having a width larger than a width of blades of the scissors and smaller than a width of handles of the scissors so that the blades of the scissors are allowed to go into the holding holes and the handles of the scissors are supported on the loading part;
a support part disposed on both lateral sides of the loading part and supporting the loading part to be disposed to be spaced apart from a ground or a surface of a piece of furniture by a predetermined distance; an internal space defined by a bottom of the loading part and the support part; and
a sterilization part configured to sterilize the blades of the scissors held in the internal space through the holding holes.

2. The scissors holder of claim 1, wherein each of the holding holes comprises:
a first holding hole having a width that defines the width of each of the holding holes which is larger than the width of the blades of the scissors; and
a second holding hole disposed above the first holding hole and having a width larger than the width of the first holding hole in order to guide the blades of the scissors through entry into the first holding hole.

3. The scissors holder of claim 2, wherein the width of the second holding hole is gradually increased from a bottom portion thereof toward a top portion thereof in order to guide the blades of the scissors through entry into the first holding in a closed status, the bottom portion of the second holding hole being connected to the first holding hole.

4. The scissors holder of claim 1, wherein the sterilization part comprises:
one or more first radiation units disposed in the internal space on a bottom surface of the loading part to sterilize the blades of the scissors, and configured to radiate ultraviolet (UV) rays; and
one or more reflectors disposed on the bottom surface of the loading part having a desired angle toward the internal space, the reflectors focusing light, radiated from the first radiation units, toward the internal space without leaking outside.

5. The scissors holder of claim 4, wherein each of the reflectors comprises:
a first panel extending from the bottom surface of the loading part;
a second panel extending from the first panel in a curved shape; and
a third panel extending from the second panel in a straight line shape.

6. The scissors holder of claim 5, wherein the reflectors are disposed on front and rear sides of the bottom surface of the loading part.

7. The scissors holder of claim 6, wherein the reflectors are further disposed on both lateral sides of the bottom surface of the loading part.

8. The scissors holder of claim 1, wherein top surfaces of the support part are inclined to support the loading part at a predetermined inclined angle.

9. The scissors holder of claim 1, further comprising a second radiation unit disposed above the loading part and attached to one side of the loading part, the second radiation unit radiating ultraviolet rays toward the scissors supported on the loading part to sterilize the handles of the scissors.

10. The scissors holder of claim 4, wherein the first radiation units radiate ultraviolet C (UV-C) light in a wavelength range of 100 to 280 nm.

11. The scissors holder of claim 9, wherein the second radiation unit radiates ultraviolet C (UV-C) light in a wavelength range of 100 to 280 nm.

12. The scissors holder of claim 11, wherein the second radiation unit includes a variable portion selectively changeable in a shape to adjust a position of the second radiation unit.

13. The scissors holder of claim 12, wherein the second radiation unit comprises a reflector having a desired angle toward a light source disposed inside the second radiation unit, and the reflector of the second radiation unit focuses light, radiated from the second radiation unit, toward the handles of the scissors without leaking outside.

14. The scissors holder of claim 13, wherein the second radiation unit is coupled to the loading part to be freely detachable therefrom.

15. The scissors holder of claim 1, wherein an ultraviolet blocking coating is applied to surfaces of the support part.

16. A scissors holder having a sterilization function, the scissors holder comprising:
a loading part configured to hold scissors, the loading part including holding holes each having a width larger than a width of blades of the scissors and smaller than a width of handles of the scissors so that the blades of the scissors are allowed to go into the holding holes and the handles of the scissors are supported on the loading part;
a support part disposed on both lateral sides of the loading part and supporting the loading part to be disposed to be spaced apart from a ground or a surface of a piece of furniture by a predetermined distance;
an internal space defined by a bottom of the loading part and the support part;
a first radiation unit disposed in the internal space radiating ultraviolet rays to sterilize the blades of the scissors held in the internal space through the holding holes; and
a second radiation unit disposed above the loading part and attached to one side of the loading part, the second radiation unit radiating ultraviolet rays toward the scissors supported on the loading part to sterilize the handles of the scissors.
